(19) United States
(12) Patent Application Publication
(10) Pub. No.: US 2008/0219923 A1
(43) Pub. Date: Sep. 11, 2008

(54) Method and Kit for the Production of Particles Labelled with Rhenium-188

(75) Inventors: Gerd Wunderlich, Dresden (DE); Antje Drews, Dresden (DE)

Correspondence Address: GUDRUN E. HUCKETT DRAUDT, SCHUBERTSTR. 15A, WUPPERTAL, 42289, DE

(73) Assignee: ROTOP PHARMAKA GMBH, Radeberg (DE)

(21) Appl. No.: 10/597,092

(22) PCT Filed: Jan. 27, 2005
(86) PCT No.: PCT/DE05/00140

§ 371 Date: Aug. 28, 2006

(30) Foreign Application Priority Data
Jan. 29, 2004 (DE) 10 2004 005 280.8

(51) Int. Cl. A61K 51/08; A61K 51/00; A61P 35/00

(52) U.S. Cl. 424/1.69; 424/1.65

(57) ABSTRACT

Rhenium-188 labeled particles are produced by suspending particles of an organic polymer or a biopolymer in a solution wherein the solution contains a water-soluble tin-II salt and a Re-188 perrhenate salt with a radioactivity of 1,000 MBq to 60,000 MBq and wherein the solution has initially a pH value of pH 1 to pH 3; heating the solution of step a) to 80° C. to 100° C.; and, after 45 minutes to 70 minutes of heating, increasing and adjusting the pH value to a pH value of pH 5 to pH 8.5.

The invention relates to a method for producing particles labeled with radioactive isotope rhenium-188 (Re-188) and a kit for performing the method. Such radioactively labeled particles can be used in medicine, preferably in the field of oncology and nuclear medicine, for radiotherapy of tumors or metastases of tumors.

The radiotherapy of tumors or their metastases with radioactively labeled particles is known. In general, for this purpose a catheter is inserted into the vessels leading to the tumor. Through the catheter, the radioactively labeled particles are subsequently supplied locally to the tumor tissue. The radioactively labeled particles have a size that guarantees that they get stuck when first passing the tumor-infiltrating capillary blood system in the capillaries of the tumor. The method makes it possible to reach very high radioactive doses in the targeted tumor tissue while at the same time the surrounding tissue or other organs of the patient are protected. Significantly higher radiation doses in the tumor tissue have been achieved in comparison to e.g. systemic intravenous application of radioactively labeled antibodies, peptides and other low-molecular compounds.

In the last decade, primarily radionuclides Y-90, Re-188 and Ho-166 have been used for labeling appropriate particles. The beta ray emitter Re-188 with relatively minimal half-life of 17 hours is especially suitable for a therapy with high radionuclide doses and several applications to the same patient.

However, the current labeling methods for Re-188 and particles are unsatisfactory.

Labeling methods that can be effectively performed in related chemical elements, for example, technetium, are not transferable onto labeling with Re-188 as a result of the different chemical properties, in particular, the different redox potentials.

Preferred as a carrier material in nuclear medicine for radionuclide transport are human serum albumin microspheres of an average particle size of 20 micrometers ([^{99m}Tc] HSA microspheres B20, Rotop Pharmaka, Germany; Wunderlich G. et al. Applied Radiation and Isotopes 52 (2000), pages 63-68). These protein particles are degradable within the organism so that the microspheres only temporarily clog the capillaries and can be infused several times to the patient. When the labeling method developed for technetium is used for labeling Re-188, labeling yields of only less than 5% are achieved as a result of the differences in the redox potential.

A disadvantage of the method described in Wunderlich et al. is that after more than 90 min. reaction time only 70% to maximally 90% of Re-188 is bonded to the particles. In order to prevent that unreacted Re-188 causes undesirable radiation exposure in the organism of the patient, it is necessary to remove the excess Re-188 by several washing steps. These washing steps require a direct handling of radioactive liquids and therefore cause a high radiation exposure of the personnel.

Publications by Wang S. J. et al. (Journal of Nuclear Medicine 1998, 39 (10), pp. 1752-1757, Nuclear Medicine Communications 1998, 19: pp. 427-433) also disclose methods for labeling microspheres with Re-188. These microspheres are comprised of plastic resin. Disadvantageously, in these methods the microspheres after labeling with Re-188 must also be washed by removing the supernatant and resuspending in saline solution. In this method, for labeling 20 mg microspheres 200 mg tin salt and a highly acidic pH value are required. The great amount of tin has the disadvantage that the patient is additionally pharmacologically exposed. Because of the highly acidic pH value, the method is not suitable for protein particles because the protein would be hydrolyzed by the employed highly acidic 0.2 N HCl.

Grillenberger K. G. et al. disclose Re-188 labeled hydroxy apatite and sulfur colloid (Nuclear Medicine 1997, 36: pp. 71-75). The yield obtained by this labeling method is however less than 80%.

The known methods for labeling particles with Re-188 are time-consuming. The obtained labeling yields are greatly dependent on the employed base material.

There is a need in nuclear medicine for a method that simplifies labeling of particles with Re-188 for hospital personnel. The handling of high radioactive doses of rhenium-188 should be as short as possible in order to keep the radiation exposure of the personnel within an acceptable range. A method that can eliminate washing steps would therefore be desirable.

It is an object of the invention to provide a simplified method for labeling particles with rhenium-188 as well as a kit for performing the method. In particular, the method and the kit should reduce the radiation exposure of the personnel and the required time for performing the method.

According to the invention, the object is solved by a method for producing rhenium-188 (Re-188) labeled particles in which method the particles are first suspended in an acidic solution and heated and, after a certain amount of time of heating, the pH value is increased.

The solution has in this connection a pH value of pH 1 to pH 3 and contains:
a) a tin-II salt and
b) a Re-188 perrhenate salt.

The preferred reaction volume in this step is 1 to 5 ml, especially preferred 2 ml to 4 ml, especially advantageously 3 ml. The known Re generators deliver a minimum eluate volume of 2 ml. Advantageously, by means of the reaction volume in the method according to the invention the entire eluate of a Re generator can be utilized.

After 30 to 240 minutes, preferably 45 to 70 minutes, of heating, the pH value is increased. In this connection, the pH value is adjusted to be greater than pH 5, preferably between pH 6.5 to pH 8.5.

Surprisingly, the yield of labeling the particles with Re-188 is increased to more than 95% by increasing the pH value at the end of heating. By the thus obtained effective labeling of particles with Re-188 a further processing of the end product is no longer required. In particular, washing steps are no longer needed. The suspension obtained by increasing the pH values can be directly used for radiotherapy of the patient.

The total reaction time is shortened significantly in comparison to the prior art. By eliminating washing steps, in addition to saving time the radiation protection for the personnel is significantly improved because fewer manipulations are required in order to arrive at an injectable product.

By means of the method a specific radioactivity (labeling of the particles) is reached that is significantly above the labeling that has been described before by Wunderlich et al. (2001): 2,500 MBq/mg in comparison to 500 MBq/mg. The increase of the pH value is realized by adding a buffer solution, preferably acetate, citrate, or tartrate solution, especially preferred a potassium sodium tartrate solution.

The buffer solution after having been added to the heated solution preferably has a final concentration of 15 mmol/l to 50 mmol/l, particular preferred 25 mmol/l.

The tin-II salt is preferably a water soluble tin-II salt, for example, SnCl₂×2H₂O or SnF₂, which at the beginning of the method is present in the solution in a concentration of 10 mmol/l to 50 mmol/l, especially preferred 17 mmol/l.

By means of the method, the Re-188 initially present as perrhenate (ReO₄⁻) in the oxidation state +VII is reduced by the reductive effect of the tin-II salt. In this way, the oxide of Re-188 is precipitated in the oxidation state +4 (ReO₂×H₂O) together with the generated sparingly soluble tin hydroxide on the microspheres. The resulting layer generated by co-precipitation has a thickness of approximately 1 μm.

With the method according to the invention, the amount of the tin-II salt required for labeling can be reduced by a factor 10 in comparison to the prior art (Wang et al.). An amount of 10 mg to 12 mg of tin(II) salt per 10 mg microspheres has surprisingly been found to be sufficient for labeling the microspheres.

Since tin-II salts are relatively instable in aqueous solution when heated, a complexing agent for stabilizing the tin-II salt is added to the solution. Such a complexing agent is preferably an organic carboxylic acid, especially preferred 2,5-dihydroxy benzoic acid (gentisic acid). Further preferred complexing agents are acetic acid, citric acidic, malonic acid, gluconic acid, lactic acid, hydroxy isobutyric acid, ascorbic acid, tartaric acid, succinic acid, the salts of the aforementioned acid, or glucoheptonate. The complexing agent for stabilizing the tin-II salt has in solution preferably a concentration of 50 mmol/l to 30 mmol/l, particularly preferred 20 mmol/l.

The use of gentisic acid is advantageous because gentisic acid is a radical scavenger and therefore acts as a radiation-protective agent in the preparation. Gentisic acid, moreover, is already approved as an additive for pharmaceuticals.

Heating of the solution is realized preferably to a temperature below boiling point, in a range of 80° C. to 100° C.

The particles to be labeled are preferably spherical or approximately round. Such particles, referred to as microspheres, have advantageously a diameter that is small enough so that the microspheres can be transported through normal blood vessels but large enough that they get stuck in the capillaries. Preferably, they have a diameter of 10 μm to 100 μm, especially preferred 15 μm to 30 μm.

The particles are preferably comprised of an organic polymer or a biopolymer. In one embodiment of the invention, the particles are comprised of a polymer that cannot be degraded in vivo, preferably a weak cation exchange resin (e.g. Bio-Rex 70, BioRad, Germany), polyacrylate, polymethylmethacrylate (PMMA, e.g. Heraeus Kulzer, Germany), methacrylate copolymer (e.g. MacroPrep, BioRad, Germany) or polyvinyl formaldehyde (e.g. Drivalon, Nycomed-Amersham, Germany).

Particularly preferred particles are however microspheres of a material that can be metabolized and degraded in the human organism so that the particles will clog the capillaries after application only temporarily. Advantageously, this enables several applications of the particles. A preferred example of such degradable particles are microspheres of human serum albumin ([^{99m}Tc] HSA microspheres B20, Rotop Pharmaka, Radeberg, Germany). The [^{99m}Tc] HSA microspheres B20 are already approved for use with labeling by technetium 99m.

Comparative examples with particles of different biodegradable materials have shown that with microspheres of human serum albumin in the method according to the invention surprisingly a significantly higher Re-188 labeling yield and greater in vivo stability can be achieved than with other materials that are also degradable in vivo. For example, the labeling yield with particles of macro-aggregated albumin (MAA, Nycomed-Amersham, Germany), collagen particles (Angiostat, Regional Therapeutics, USA) and polyacetate particles (PLA, Micromod, Germany) is significantly lower.

The particles during labeling are preferably present in a concentration of 2 to 3 million particles, preferably 2.5 million particles, per milliliter, or 0.5 to 10 million particles per milliliter.

The beta ray emitter rhenium-188 used for labeling is practically available in unlimited quantities over several months after purchasing a corresponding radionuclide generator (Oak Ridge National Laboratory, TN, USA, or Schering AG, Germany) and is suitable in particular for a therapy with high radionuclide doses and several applications to the same patient. In such a generator, the Re-188 is eluated in the form of perrhenate (oxidation state VII of Re-188) by applying an 0.9% saline solution. The thus obtained Re-188 generator eluate has preferably a radioactivity of 1,000 MBq to 60,000 MBq, preferably of 10,000 to 20,000 MBq.

The specific radioactivity (labeling of the particles) obtained with the method according to the invention is preferably 1,500 to 3,000 MBq/mg. Advantageously, the specific radioactivity can be adjusted in regard to the patient to the desired therapeutic radiation dose by the employed amount of Re-188 generator eluate.

Advantageously, the method according to the invention is therefore suitable for labeling microspheres with radioactivities that are within the therapeutic range. Because of this and because of the aforementioned simplification of the method steps, the development of a pharmaceutical kit is advantageously possible.

A further object of the invention is a pharmaceutical kit for performing the method according to the invention. This kit for producing rhenium-188 labeled microspheres comprises the following components:
a) a container with a quantity of water-soluble tin-II salt and a complexing agent stabilizing the tin-II salt, each in a powder form or as a solution,
b) a second container with microspheres of human serum albumin, as well as
c) a third container with a substance or solution for increasing the pH value, in powder form or as a solution.

The substance for increasing the pH value is present in solid form or aqueous solution and results in solution in a pH value of pH 6.5 to pH 8.5.

Preferably, the components are distributed onto different containers. The kit contains in this embodiment at least one of the three containers per administration to the patient.

In an especially advantageous configuration of the invention, acetate, citrate or tartrate, preferably potassium sodium tartrate, is used for increasing the pH value. For each administration to the patient, the kit contains preferably 0.1 mmol to 0.2 mmol of a substance for increasing the pH value, especially preferred 30 mg to 50 mg potassium sodium tartrate×4H₂O.

The tin-II salt is preferably a water-soluble tin-II salt, for example, tin(II) chloride dihydrate or SnF₂. For each administration to the patient, the kit contains preferably 0.02 mmol to 0.1 mmol of the water-soluble tin-II salt, especially preferred 5 mg to 20 mg tin(II)chloride dihydrate.

Because the tin-II salts in aqueous solution are relatively instable when heated, the kit contains preferably as a further component a complexing agent for stabilizing the tin-II salts. Such a complexing agent is preferably an organic carboxylic acid or a salt of an organic carboxylic acid. The complexing agent is contained in the container (a) with the tin-II salt.

An especially preferred complexing agent for stabilizing tin-II salt is 2,5-dihydroxy benzoic acid (gentisic acid). Further preferred complexing agents are acetate, citrate, malonate, gluconate, malate, lactate, hydroxy isobutyrate, pyrophosphate, ascorbate, potassium sodium tartrate or glucoheptonate. For each application to the patient the kit contains preferably 0.5 to 2 mol, in particular preferably 1 mol, of the complexing agent stabilizing the tin-II salt per mol tin-II salt. This corresponds to a quantity of 5 mg to 20 mg gentisic acid.

The kit contains as further components also the particles to be labeled. These particles are preferably round or approximately round. Such particles, microspheres, have advantageously a diameter that is small enough that the microspheres can be transported through regular blood vessels but large enough to get caught in capillaries. Preferably, they have a diameter of 10 μm to 50 μm, especially preferred 10 μm to 30 μm.

The kit contains preferably 0.5 to 10 million, especially preferred 1 to 5 million particles, advantageously 1 to 2 million in an additional container (b).

The particles are comprised preferably of a material that is metabolized and degraded in the human organism such that these particles will clog the capillaries upon administration only temporarily. Advantageously, in this way multiple applications of the particles are possible. A preferred example of such degradable particles are microspheres of human serum albumin ([^{99m}Tc] HSA microspheres B20, Rotop Pharmaka, Radeberg, Germany). The [^{99m}Tc] HSA microspheres B20 are already approved for use with labeling by technetium 99m.

The particles are contained in the kit preferably in a concentrated aqueous or alcoholic suspension. In order to increase the dispersion of the particles, advantageously a non-ionic detergent is added to this suspension. Preferably, non-ionic detergents of the polyethylene type, for example, polyoxyethylene sorbitan monooleate (Tween® 80), are used.

The non-ionic detergent is preferably contained in an amount of 0.15 mg to 0.3 mg per 1 mg particle in the suspension.

For producing Re-188 labeled microspheres, the tin-II salt and the complexing agent for stabilizing the tin-II salt are dissolved in the first container in sterile water and added to the second container containing the microspheres and the microspheres are suspended in the solution. The generator eluate containing the radioactive rhenium-188 is added to the suspension and the suspension is heated to 80° C. to 100° C. After 45 minutes to 70 minutes of heating, the pH value is adjusted to pH 5 to pH 8.5 by mixing the suspension with the substance for increasing the pH value that is contained in the third container. The suspension is now cooled, preferably to body temperature, and can be administered without washing steps directly to the patient.

The invention also concerns the particles produced with the method according to the invention and the kit according to the invention and their use for radiotherapy of carcinoma or their metastases.

A further component of the invention is a method for radiotherapy of tumors, carcinoma or their metastases with these particles. In the method, by means of the method as described above particles labeled with Re-188 are produced. Into the local blood vessel that leads to the carcinoma a catheter is inserted. Through the catheter, a suspension of the radioactively labeled particles, after adjusting the pH value to pH 5 to pH 8.5, is subsequently supplied locally to the tumor tissue (without intermediate washing of the particles). The radioactively labeled particles have a size that ensures that upon the first passage of the tumor-infiltrating capillary blood system they remain within the capillaries of the tumor. Preferably, the particles have for this purpose a diameter of 10 μm to 50 μm, particularly preferred 10 μm to 30 μm.

The method enables advantageously that very high radioactivity doses are reached in the targeted tumor tissue while at the same time the surrounding tissue and other 30 organs of the patient are protected. Significantly higher radiation doses (100-150 Gy) in the tumor tissue are achieved in comparison to, for example, systemic intravenous administration of radioactively labeled antibodies, peptides and other low-molecular compounds.

The use of microspheres of human serum albumin has the advantage that the particles can be degraded in the body. The microspheres close off the capillaries only temporarily when administered. Multiple administrations are thus possible.

The administration of the particles is carried out preferably arterially by means of infusion. For this purpose, preferably 0.5 to 10 million, particularly preferred 1 to 5 million particles, advantageously 1 to 2.5 million (corresponding to 1 to 20 mg, preferably 3 to 10 mg) are suspended in 20 ml to 100 ml, preferably 50 ml, of an infusion solution (for example, sterile isotonic saline solution) and infused.

The microspheres are degraded with a biological half-life in the range of preferably greater than 200 hours, preferably eight days to 15 days. The biological half-life of the microspheres is thus in the range of the biological half-life of Re-188. Advantageously, by immobilizing Re-188 on the microspheres, the Re-188 is fixed at the application location (>90% remain resident there over days).

The microspheres labeled with Re-188 in accordance with the present invention are suitable advantageously in particular for the therapy of liver carcinoma and liver metastases of other carcinoma.

With the aid of the following examples the invention will be explained in more detail:

### EXAMPLE 1

Labeling of particles with Re-188 is explained with the aid of labeling of human serum albumin (HSA) microspheres as follows:

9.3 mg 2,5-dihydroxy benzoic acid (gentisic acid) are dissolved in 2 ml water for injection, subsequently 11.4 mg SnCl₂×H₂O are added, and the solution is sterilely filtered into a bottle containing human serum albumin (HSA) microspheres (MS B20, Rotop Pharmaka, Radeberg, Germany). The particles in the bottles are slurried and transferred into another kit bottle MS B 20 and subsequently into a third kit bottle. In the third bottle 1.5 million particles MS B 20 are then contained. To this is added 1 ml sterilely filtered Re-188 perrhenate (10,000-20,000 MBq) dissolved in 0.9% NaCl. The kit bottle with the particles is then inserted into a heating block and the latter is shaken for 55 minutes at 95° C. Subsequently, 0.6 ml sterilely filtered KNa tartrate solution (42 mg/ml) are added and heating is switched off. After five minutes of additional shaking, the preparation is ready to be injected.

The labeling yield (radiochemical purity) of the particles labeled in this way is than 95%.

### EXAMPLE 2

A preferred kit for labeling particles (in this case: human serum albumin (HSA) microspheres) with Re-188 is comprised of three flasks with the ingredients listed in Table 1.

TABLE 1

| bottle | component | quantity/bottle | | process | consistency |
|---|---|---|---|---|---|
| 1 | 2,5 dihydroxy benzoic acid | 9.3 | mg | lyophilized | solid |
| | tin(II)chloride dihydrate | 11.4 | mg | | |
| | ultra high purity nitrogen 5.0 | | | | |
| 2 | HSA microspheres A20 | 10 mg (1.2 × | | vacuum- | solid |
| | (diameter 10-30 μm) | 10⁶ to 2 × 10⁶ | | concentrated | |
| | | particles) | | | |
| | Tween ® 80 | 2.4 | mg | | |
| | ultra high purity nitrogen 5.0 | | | | |
| 3 | potassium-sodium tartrate | 1 | ml | sterilized | liquid |
| | solution (42 mg/ml) | | | | |

** ultra high purity nitrogen is used as an inert gas

The kit is designed for the treatment of a patient.

### EXAMPLE 3

With the kit according to Example 2 the particles (in this case: human serum albumin (HSA) microspheres) are labeled according to the following labeling procedure:

The components of the kit bottle 1 (2,5-dihydroxy benzoic acid—gentisic acid) and tin(II) chloride dehydrate) are dissolved in 2 ml sterile pyrogen-free water for injection purposes and added in the kit bottle 2 to the HSA microspheres A20. After adding the solution, for pressure compensation the same volume of nitrogen is to be removed with a syringe from the bottles 1 and 2. By slight shaking causing wetting of the rubber lyo stopper the HSA microspheres are suspended.

[¹⁸⁸Re] sodium perrhenate in sterile, isotonic pyrogen-free sodium chloride solution (¹⁸⁸Re generator eluate (10,000-20,000 MBq), volume: 1 ml) is transferred into the bottle 2 which is arranged in a lead shielding. After adding the ¹⁸⁸Re generator eluate for pressure compensation the same volume of nitrogen is to be removed from the bottle 2.

For carrying out the reaction, the bottle 2 is shaken in a heater/shaker for 55 minutes at 95° C. The bottle 2 is removed from the shaker and 0.6 ml of the bottle 3 (K/Na tartrate solution) is transferred into bottle 2. After adding the solution, for pressure compensation the same volume of nitrogen is to be removed from bottle 2. By slight shaking with wetting of the rubber lyo stopper the [¹⁸⁸Re] HSA microspheres are suspended.

The preparation in the bottles is allowed to react for 5 more minutes at room temperature by using the shaker; the preparation is then ready to be injected. The suspension of the labeled [¹⁸⁸Re] HSA microspheres B20, depending on the desired concentration, can be diluted with sodium chloride solution for injection. The [¹⁸⁸Re] HSA microsphere suspension can be used up to two hours after labeling.

### EXAMPLE 4

The kit according to Example 2 is produced as follows:

For a batch of 150 bottles No. 1.395 g gentisic acid (2.5-dihydroxy benzoic acid) and 1.710 g of tin(II) chloride dihydrate are dissolved in 150 ml water for injection purposes. The solution is distributed onto 150 bottles and lyophilized.

For a batch of 200 bottles No. 2, 2.0 g HSA microspheres A20 (Rotop Pharmaka GmbH, Germany) and 0.48 g Tween® 80 are suspended in a solution of:
360 mm acetone
40 ml sodium hydroxide solution (0.1 mol/l)
40 mm hydrochloric add (0.1 mol/l
240 ml ethanol abs.

To the suspension a minimal amount of the dye bengal pink is added. The suspension is concentrated in vacuum to 400 ml and distributed onto the 200 bottles. Subsequently, acetone and ethanol are removed by vacuum drying.

For a batch of 150 bottles No. 3, 6.3 g potassium sodium tartrate are dissolved in 150 ml water for injection purposes. The solution is distributed onto 150 bottles.

### EXAMPLE 5

In accordance with the procedure of Example 1, particles of different materials were labeled with Re-188:

S1 weak polyacrylate cation exchange resin (Bio-Rex 70, BioRad, Germany),
S2 polymethylmethacrylate (PMMA, Heraeus Kulzer, Germany)
S3 methacrylate copolymer (MarcoPrep Q, BioRad, Germany)
S4 polyvinyl formaldehyde (Drivalon, Nycomed-Amersham, Germany)
S5 macro-aggregated albumin (MM, Nycomed-Amersham, Germany)
S6 human serum albumin (HSA B20, ROTOP Pharmaka GmbH, Germany)
S7 collagen particles (Angiostat, Regional Therapeutics, USA),
S8 polylactate particles (PLA, Micromod, Germany).

2-3 mg of the particles (corresponding to approximately 0.5 million particles) were used, respectively.

Before and after labeling, the distribution of the particle size was determined according to ISO 13323-1 by means of single-particle light scattering. After dilution in particle-free water the particles were measured sequentially in the measuring zone of the flow cuvette. The size distribution of the particles was recalculated according to ISO 9276-2 into a surface area-based distribution because this better characterizes the distribution of Re-188 on the labeled particle surface.

In Table 2 the values of the cumulative distribution (Q₂) according to ISO 1998 is provided; the values represent 90% of the surface area-based total distribution.

The labeling yield was determined after labeling by centrifugation of the particle suspensions and radioactivity measurement of the supernatant and precipitate in an automated gamma counter (Cobra II, Packard, USA).

TABLE 2

| | | | particle size | particle | |
|---|---|---|---|---|---|
| | | | before | size after | labeling |
| | | | labeling | labeling | yield |
| | material | | [μm] | [μm] | [%] |
| S1 | Biorex 70 | macro reticular acrylic | 45-75 | 30-75 | 80-85 |
| | | resin | | | |
| S2 | PMMA | polymethylmethacrylate | 4-25 | 4-25 | 70-85 |
| S3 | Macro Prep | methacrylate Copolymer | 45-55 | 30-80 | 83-90 |
| S4 | Drivalon | polyvinyl formaldehyde | 50-150 | 5-150 | 60-70 |
| S5 | MAA | human serum albumin | 10-100 | 10-50 | 60-70 |
| S6 | HSA B20 | human serum albumin | 13-27 | 15-37 | 95* |
| S7 | Angiostat | collagen | 20-75 | 1-15 | 35-50 |
| S8 | PLA | polylactate | 10-45 | 3-45 | 50-60 |

After labeling with Re-188 the biodegradable HSA microspheres B20 (under the microscope recognizable as spheres) had a hardly changed distribution between 15 μm and 37 μm (average value 21 μm).

In contrast to this, the macro-aggregated HSA (MAA) after labeling had a broad particle distribution. This is caused by MAA particles not being present as round microspheres but having irregularities similar to little sponges. MAA particles are not so stable at high temperatures and, because of the greater surface area, are also much faster attacked and degraded enzymatically in vivo.

Drivalon (S4), Angiostat (S7) and PLA (S8) particles also do not survive well the labeling process at the required high reaction temperature i.e., there is increased fine material and the particle distribution is broadened significantly. Despite of this, labeling for all particle preparations in vitro is rather stable.

In order to test the in vitro stability, the labeled particle samples were incubated with human plasma. After three hours of incubation at 37° C. or after 24 h and 48 h incubation at room temperature, the adhesion of Re-188 on the particles after centrifugation and radioactivity measurement was determined in an automated gamma counter (Cobra II, Packard, USA).

The results of in vitro stability of the labeled particles are summarized in Table 3.

TABLE 3

| | | particle-bonded radioactivity, | | | |
|---|---|---|---|---|---|
| | | average value ± standard deviation (SD) [%] | | | |
| | | | t = 3 | t = 24 | t = 48 |
| | material | t = 0 | h/37° C. | h/22° C. | h/22° C. |
| S1 | Biorex 70 | 100 | 93.3 ± 2.3 | 92.3 ± 1.6 | 86.3 ± 3.5 |
| S2 | PMMA | 100 | 95.2 ± 1.7 | 93.8 ± 2.4 | 91.3 ± 2.7 |
| S3 | Macro Prep | 100 | 92.7 ± 3.4 | 83.5 ± 1.6 | 82.1 ± 2.8 |
| S4 | Drivalon | 100 | 95.0 ± 2.5 | 84.4 ± 3.4 | 79.9 ± 3.5 |
| S5 | MAA | 100 | 97.3 ± 2.0 | 92.1 ± 1.7 | 86.3 ± 3.1 |
| S6 | HSA B20 | 100 | 98.0 ± 1.8 | 92.2 ± 2.2 | 86.8 ± 2.4 |
| S7 | Angiostat | 100 | 92.0 ± 3.4 | 85.2 ± 3.2 | 82.8 ± 1.6 |
| S8 | PLA | 100 | 96.1 ± 2.7 | 80.4 ± 2.8 | 75.7 ± 1.9 |

The in vitro stability of all particle preparations can be considered to be satisfactory because 75-90% of Re-188 after 48 hours is still particle-bonded (Table 3).

The biodistribution of the different particles was examined in vivo after intravenous injection in Wistar rats, wherein the lungs served as a model for a tumor that has a good blood supply.

After injection of particles labeled with 20 MBq Re-188 the biodistribution of the particles was examined over 48 hours under gamma camera (Picker CX 250) with the aid of conventional nuclear-medical imaging technology. At the end of the gamma camera examinations, the animals were killed, select organs removed, and their radioactivity determined in a gamma counter in comparison to the entire animal and to the injected activity.

The in vivo biodistribution in the liver and the lungs (in % of injected doses in the entire organ, respectively) of the labeled particles was determined 48 h after injection into the tail vein of 8-week old Wistar rats (n=3 to 6) for each material.

In order to determine the in vivo stability of the different particle preparations, the biological half-life in the lungs (T_{b 1/2}) was used as a gauge and delivered values between 45 hours up to more than 200 hours. A biological half-life of 200 hours corresponds in this connection to an effective half-life of 15.4 hours for Re-188. Since after five effective half-lives (i.e., 77 hours) only approximately 3% of the initial radioactivity is present in the body and can act therapeutically, the obtained stability can be considered to be satisfactory.

The results of in vivo biodistribution and in vivo stability are summarized in Table 4.

TABLE 4

| | | liver | liver | |
|---|---|---|---|---|
| | | (% injected | (% injected | |
| | material | dosage) | dosage) | T_{b 1/2} [b] |
| S1 | Biorex 70 | 3.9 | 91.4 | >200 |
| S2 | PMMA | 16.7 | 76.4 | >200 |
| S3 | Macro Prep | 0.9 | 85.1 | >200 |
| S4 | Drivalon | 56.5 | 19.6 | 125.3 |
| S5 | MAA | 2.8 | 48.0 | 45.4 |
| S6 | HSA B20 | 0.8 | 92.9 | >200 |
| S7 | Angiostat | 49.6 | 14.5 | 129.7 |
| S8 | PLA | 11.1 | 66.5 | 153.9 |

The bio distribution studies show very good in vivo stability for the preparations S1 to S3 and S6, characterized by a very slow drop of radioactivity in the lungs and a minimal radioactivity uptake in the non-target tissues (for example, the liver, except in the case of S2—Table 4). S2 has already in the base material a relatively high proportion of fine particles that leads to particle deposition in the liver where the particles however stay for the duration of the experiment and remain unchanged.

Small particles (<10 μm, as, for example, sample S2) are collected in reticulo-endothelial system (RES) in liver and spleen. When fine material is generated in the labeling process, it is found after intravenous (iv) injection in these organs (sample S4, S7, and S8). These particles are therefore not suitable for intra-arterial tumor therapy in humans even though the biological half-life is relatively long (>120 h).

MAA macro-aggregates (S5) are not suitable for intra-arterial tumor therapy in humans because of their relatively minimal biological half-life (45.4 h).

In comparison to the fatal medical results reported by Mantravadi 1981 (Mantravadi R V, Spigos D G, Tan W S, Felix E L, Intraarterial yttrium-90 in the treatment of hepatic malignancies; Radiology 1981; 142: 783-786) when employing a long-lived beta emitter (Y-90) not satisfactorily bonded to the particles, the application of particles labeled with short-lived emitter Re-188 is significantly less dangerous. Re-188 released by the particles is not accumulated in vitally important organs but in a short period of time is excreted via the kidneys.

A further advantage of the use of Re-188 preparations is that the available radionuclide generator can be employed at any time for producing Re-188 preparations so that a request by a physician can be responded to without a long waiting period and at attractive costs.

The results of the comparative examples with different particle materials can be summarized as follows:

With the method according to the invention different particle materials can be labeled in high yield with Re-188 and used in a promising way for endo-radiotherapeutic applications.

HSA microspheres B20 labeled with Re-188 are the most attractive nuclear medical therapeutic agent for a local tumor therapy after selective catheter placement in the supplying blood vessels, in particular because of the biocompatibility of the particles, their uniform size, and because of the high in vivo stability of the product.

What is claimed is:

1-15. (canceled)

16. A method for producing rhenium-188 labeled particles, the method comprising the steps of:
a) suspending particles of an organic polymer or a biopolymer in a solution wherein the solution contains a water-soluble tin-II salt and a Re-188 perrhenate salt with a radioactivity of 1,000 MBq to 60,000 MBq and wherein the solution has initially a pH value of pH 1 to pH 3;
b) heating the solution of step a) to 80° C. to 100° C.,
c) after 45 minutes to 70 minutes of heating, increasing and adjusting the pH value to a pH value of pH 5 to pH 8.5.

17. The method according to claim 16, wherein in step c) a solution of citrate, acetate, or tartrate is used for increasing the pH value.

18. The method according to claim 16, wherein in step c) a solution of potassium sodium tartrate is used.

19. The method according to claim 16, wherein the solution of step a) contains a complexing agent for stabilizing the tin-II salt, wherein the complexing agent is selected from 2, 5-dihydroxy benzoic acid acetic acid, citric acid, malonic acid, gluconic acid, lactic acid, hydroxy isobutyric acid, ascorbic acid, tartaric acid, succinic acid, salts of said acids, or glucoheptonate.

20. The method according to claim 16, wherein the solution of step a) contains 2,5-dihydroxy benzoic acid as a complexing agent for stabilizing the tin-II salt.

21. The method according to claim 16, wherein the particles have a diameter of 10 μm to 30 μm.

22. The method according to claim 16, wherein initially the water-soluble tin-II salt is present in the solution of step a) in a concentration of 10 mmol/l to 50 mmol/l.

23. The method according to claim 16, wherein the particles are comprised of human serum albumin.

24. A pharmaceutical kit for producing particles labeled with Re-188, the kit comprising:
a) a first container containing a quantity of water soluble tin-II salt and a quantity of a complexing agent for stabilizing the tin-II salt, the complexing agent selected from 2,5-dihydroxy benzoic acid, acetic add, citric acid, malonic acid, gluconic acid, lactic acid, hydroxy isobutyric acid, ascorbic acid, tartaric acid, succinic acid, salts of said acids, or glucoheptonate;
b) a second container with particles made from an organic polymer or a biopolymer;
c) a third container containing a quantity of a substance for increasing the pH value, the substance selected from citrate, acetate, or tartrate, present in solid form or in aqueous solution and generating in solution a pH value of pH 6.5 to pH 8.5.

25. The pharmaceutical kit according to claim 24, wherein the complexing agent for stabilizing the tin-II salt is 2,5-dihydroxy benzoic acid.

26. The pharmaceutical kit according to claim 24, wherein the substance for increasing the pH value is potassium sodium tartrate.

27. The pharmaceutical kit according to claim 24, wherein the particles have a diameter of 10 μm to 30 μm.

28. The pharmaceutical kit according to claim 24, wherein the kit contains 0.02 mmol to 0.1 mmol of the tin-II salt per administration to the patent.

29. The pharmaceutical kit according to claim 24, wherein the particles are comprised of human serum albumin.

30. Rhenium-188 labeled particles produced by the method according to claim 16.

31. Rhenium-188 labeled particles according to claim 30 as a radiotherapeutic agent for treating tumors, carcinoma or their metastases.
